(19) United States
(12) Patent Application Publication
(10) Pub. No.: US 2011/0171300 A1
(43) Pub. Date: Jul. 14, 2011

(54) DELTA 9 TETRAHYDROCANNABINOL DERIVATIVES

(75) Inventors: Parveen Bhatarah, Hertfordshire (GB); Kevin John Batchelor, Hertfordshire (GB); Derek Mchattie, Hertfordshire (GB); Alan Kenneth Greenwood, Hertfordshire (GB)

(73) Assignee: RESOLUTION CHEMICALS LIMITED, STEVENAGE, HERTFORDSHIRE (GB)

(21) Appl. No.: 12/527,183

(22) PCT Filed: Feb. 14, 2008
(86) PCT No.: PCT/GB08/00516

§ 371 Date: Jun. 29, 2010

(30) Foreign Application Priority Data
Feb. 14, 2007 (GB) 0702895.4

(51) Int. Cl. A61K 9/64; C07D 311/80; A61K 31/353; A61P 1/08

(52) U.S. Cl. 424/456; 549/390; 514/455

(57) ABSTRACT

A compound of formula (I)

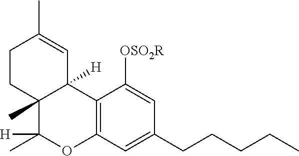

wherein R is selected from
(i) a 5 to 7 membered heterocyclic ring containing 1 to 3 heteroatoms independently selected from nitrogen, oxygen and sulfur, which is optionally substituted by one or more groups independently selected from halo, CN, NO₂, (C₁-C₆)alkyl, (C₁-C₆)alkoxy and phenyl;
(ii) (C₁-C₆)alkyl, which is optionally substituted by one or more substituents each independently selected from halo, oxo, CN, NO₂, (C₁-C₆)alkyl, (C₁-C₆)alkoxy and phenyl;
(iii) (C₁-C₆)alkyl substituted by (C₃-C₁₃)cycloalkyl, which is optionally substituted by one or more substituents each independently selected from halo, oxo, CN, NO₂, (C₁-C₆)alkyl, (C₁-C₆)alkoxy and phenyl; and
(iv) (C₃-C₁₃)cycloalkyl, which is optionally substituted by one or more substituents each independently selected from halo, oxo, CN, NO₂, (C₁-C₆)alkyl, (C₁-C₆)alkoxy and phenyl.

The present invention relates to processes for the purification of Δ9-tetrahydrocannabinol (Δ9-THC), intermediates utilised in the process, processes for their preparation and the use of the intermediates for stable storage of Δ9-THC.

Cannabinoids are a family of naturally occurring C₂₁ terpenophenolic compounds uniquely produced in cannabis. Marijuana usually refers to a mixture of leaves and flowering heads of the pistillate plant of Cannabis sativa from which tetrahydrocannabinols (THCs) are isolated. THCs contain two main isomeric forms, depending on the position of the double bond. The position of the double bond and the stereochemistry of these THCs have been confirmed by nuclear magnetic resonance and X-ray structure.

THCs have been used as psychomimetic agents for many years with the main psychomimetic activity being attributed to Δ9-THC (20 times greater than Δ8-THC).

Δ9-THC is marketed as Marinol™ and is prescribed for patients suffering from severe nausea and vomiting associated with cancer chemotherapy.

The major cannabinoids present in cannabis other than Δ8-THC and Δ9-THC are cannabinol, cannabidiol and Δ9-THC carboxylic acid which exists in two forms depending on the position of the carboxylate group. Cannabidiol may be present in cannabis in large amounts but has little activity.

The major component of cannabis is Δ9-THC carboxylic acid which exists as two isomeric forms, THCA-A and THCA-B, both of which are psychomimetically inactive.

It can be converted into the predominately active constituent Δ9-THC, slowly on storage and rapidly on exposure to heat (e.g. when smoked). In fresh, dried marijuana, 95% of cannabinoids are present as THCA-A. Only THCA-A can be readily decarboxylated to Δ9-THC due to the presence of hydrogen bonding.

It is known to extract active ingredients from cannabis plant material using ethanol or a mixture of ethanol and water. The extract typically contains large amounts of Δ9-THC and Δ9-THC carboxylic acid and the acid is converted to Δ9-THC by refluxing the cannabis extract in ethanol. Δ9-THC is then purified.

A number of difficulties exist in the purification process.

First, the USP specification for pharmaceutical compositions containing Δ9-THC, referred to as dronabinol, indicates a maximum contaminant level of cannabinoids.

The step of extracting active ingredients from cannabis routinely extracts a number of impurities which are difficult to remove from the finished product, and hence a large number of purification steps, including expensive column chromatography, are required to meet the USP requirements.

Additionally, Δ9-THC is a relatively unstable molecule that is susceptible to rearrangement and oxidation at room temperature and is therefore usually stored in a dark container at low temperature. Even under controlled conditions of storage, decomposition of the Δ9-THC takes place limiting the length of time the product can be stored.

WO 2004/043946 refers to the preparation of benzyl and naphthyl sulfonate derivatives of Δ9-THC, which are highly crystalline and stable at room temperature in air, allowing for indefinite storage. The derivatives can be recrystallised for purification and hydrolysed to recover the purified Δ9-THC.

Derivatives of, preparation of and purification of Δ9-THC are also known from WO 2004/043946, US 2006/0094774, WO 2006/133941, WO 2006/053766, U.S. Pat. No. 3,636,058 and U.S. Pat. No. 3,507,885.

There is a need for further derivatives of Δ9-THC that are stable, easily handled, easy to prepare, readily converted back to Δ9-THC, preferably using mild reaction conditions.

It is therefore the object of the present invention to provide alternative methods for the purification and storage of Δ9-THC.

It is a further object of the present invention to provide derivatives of Δ9-THC that are easy to prepare and that can be purified by standard techniques, preferably by recrystallisation. It has surprisingly been found that the compounds of the present invention are generally crystalline materials that can be prepared in high yield.

It is a further object of the present invention to provide derivatives of Δ9-THC that are readily hydrolysed in order to regenerate Δ9-THC in an efficient manner. The compounds of the present invention have the advantage of being easy to hydrolyse with the result that high recoveries of Δ9-THC are obtained containing few impurities and being easy to purify to the required level.

Accordingly, the present invention provides for compounds of formula (I)

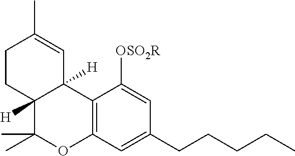

wherein R is selected from
i. a 5 to 7 membered heterocyclic ring containing 1 to 3 heteroatoms independently selected from nitrogen, oxygen and sulfur, which is optionally substituted by one or more groups independently selected from halo, CN, NO₂, (C₁-C₆)alkyl, (C₁-C₆)alkoxy and phenyl;
ii. (C₁-C₆)alkyl, which is optionally substituted by one or more substituents each independently selected from halo, oxo, CN, NO₂, (C₁-C₆)alkyl, (C₁-C₆)alkoxy and phenyl;
iii. (C₁-C₆)alkyl substituted by (C₃-C₁₃)cycloalkyl, which is optionally substituted by one or more substituents each independently selected from halo, oxo, CN, NO₂, (C₁-C₆)alkyl, (C₁-C₆)alkoxy and phenyl; and
iv. (C₃-C₁₃)cycloalkyl, which is optionally substituted by one or more substituents each independently selected from halo, oxo, CN, NO₂, (C₁-C₆)alkyl, (C₁-C₆)alkoxy and phenyl.

Unless otherwise indicated, alkyl and alkoxy groups may be straight or branched and contain 1 to 6 carbon atoms and preferably 1 to 4 carbon atoms. Examples of alkyl include methyl, ethyl, n-propyl, isopropyl, n-butyl, isobutyl, sec-butyl, pentyl and hexyl. Examples of alkoxy include methoxy, ethoxy, isopropoxy and n-butoxy.

Halo means fluoro, chloro, bromo or iodo and is preferably chloro.

A heterocycle may be saturated, partially saturated or aromatic. Examples of heterocyclic groups are tetrahydrofuranyl, pyrrolidinyl, dihydropyranyl, tetrahydropyranyl and morpholinyl. Examples of aromatic heterocyclic groups are furyl, thienyl, pyrrolyl, oxazolyl, thiazolyl, imidazolyl, pyrazolyl, isoxazolyl, isothiazolyl, oxadiazolyl, triazolyl, thiadiazolyl, pyridyl, pyrimidinyl, pyrazinyl, pyridazinyl and triazinyl.

Cycloalkyl can be monocyclic or polycyclic, including bridged systems.

Unless otherwise indicated, the term substituted means substituted by one or more defined groups. In the case where groups may be selected from a number of alternative groups, the selected groups may be the same or different.

Included within the scope of the invention are all tautomers of the compound of formula (I) and all solvates of said compound or tautomer thereof.

In a preferred aspect, the present invention comprises compounds of formula (I) wherein R is a 5 to 6 membered heteroaromatic ring containing 1 to 2 heteroatoms independently selected from nitrogen, oxygen and sulfur, which is optionally substituted by one or more groups independently selected from halo, (C₁-C₆)alkyl and phenyl.

More preferably, R is a 5 membered heteroaromatic ring containing 1 heteroatom selected from nitrogen, oxygen and sulfur, which is optionally substituted by one or more groups independently selected from halo, (C₁-C₆)alkyl and phenyl.

Yet more preferably, R is selected from thienyl, furyl and pyrrolyl, which is optionally substituted by one or more groups independently selected from halo, (C₁-C₆)alkyl and phenyl.

Even more preferably, R is selected from thienyl, furyl and pyrrolyl.

Most preferably, R is 2-thienyl.

In another preferred aspect, the present invention comprises compounds of formula (I) wherein R is (C₁-C₆)alkyl, which is optionally substituted by one or more substituents each independently selected from halo, oxo, (C₁-C₆)alkyl and phenyl.

Preferably, R is (C₁-C₆)alkyl, which is optionally substituted by one or more chloro substituents.

More preferably, R is n-propyl, which is optionally substituted by chloro.

Most preferably, R is n-propyl or 3-chloropropyl.

In another preferred aspect, the present invention comprises compounds of formula (I) wherein R is (C₃-C₁₃)cycloalkyl or (C₁-C₆)alkyl substituted by (C₃-C₁₃)cycloalkyl, each cycloalkyl being optionally substituted by one or more substituents each independently selected from halo, oxo and (C₁-C₆)alkyl.

Preferably, R is (C₃-C₁₀)cycloalkyl or methyl substituted by (C₃-C₁₀)cycloalkyl, each cycloalkyl being optionally substituted by 1 to 3 substituents each independently selected from chloro, oxo and methyl.

More preferably, R is methyl substituted by cyclopropyl, cyclobutyl, cyclopentyl, cyclohexyl, norbornyl, decalinyl or adamantyl, which is optionally substituted by 1 to 3 substituents each independently selected from chloro, oxo and methyl.

Most preferably, R is camphor.

Preferred compounds of formula (I) are:
Δ9-THC (1S)-(+)-Camphorsulfonate;
Δ9-THC 2-thiophene sulfonate;
Δ9-THC n-propyl sulfonate; and
Δ9-THC 3-chloropropyl sulfonate.

The preferred compounds of formula (I) have the advantage that they are easy to prepare, readily purified, stable to light and air at ambient temperature and readily hydrolysed in order to regenerate Δ9-THC in a high yield with high purity.

### Processes

Compounds of formula (I) wherein R is as described herein may be prepared by reacting Δ9-THC with a sulfonyl halide of formula RSO₂Cl in the presence of a base, optionally in the presence of a solvent. Suitable bases include, inter alia, triethylamine, pyridine and dimethylaminopyridine. Suitable solvents include, inter alia, toluene and dichloromethane.

The compounds of formula (I) may be purified by standard techniques known to those skilled in the art, such as recrystallisation and column chromatography. Suitable solvents for recrystallisation include methanol and heptane.

Δ9-THC may be regenerated from the compounds of formula (I) by hydrolysis, for example, in an alcohol, such as methanol, in the presence of a base, such potassium hydroxide. Other suitable solvents include, inter alia, water, ethanol, n-propanol, isopropanol, t-butanol, and mixtures of water and alcohols. Other suitable bases include, inter alia, sodium and potassium methoxides, ethoxides, n-propoxide, isopropoxides and t-butoxides. The reaction may conducted at room temperature or at higher temperatures, such as 40° C. to 80° C.

Further processes that may be used for the preparation of compounds of formula (I) and for the regeneration of Δ9-THC are disclosed in WO 2004/043946.

The invention is illustrated by the following non-limiting examples in which the following abbreviations and definitions are used.

br broad
d doublet
ESI positive mode electrospray ionisation

### HPLC High Performance Liquid Chromatography

J Coupling constant

### LCMS Liquid Chromatography Mass Spectroscopy

m multiplet
RT Room Temperatures singlet
t triplet

### TLC Thin Layer Chromatography

### wt Weight

### Vols Volumes

### EXAMPLE 1

### Δ9-THC (1S)-(+)-Camphorsulfonate

To a solution of crude Δ9-THC (5.0 g, 15.90 mmol) and Et₃N (1.77 g, 17.49 mmol) in toluene (5 vols) was added (1S)-(+)-camphorsulfonyl chloride (4.39 g, 17.49 mmol) under a N₂ blanket. TLC monitoring indicated reaction completion after stirring for 4.5 h at RT. The reaction was quenched by adding water (5 vols) and stirring for 30 min. The toluene phase was separated and further washed with water (2×5 vols), followed by saturated aqueous NaCl (1×5 vols). The organic phase was dried over MgSO₄ and filtered. The solvent was evaporated to dryness under reduced pressure. The crude residue was triturated with n-heptane (10 vols) to induce crystallisation. The white slurry was further stirred at 0° C. for 60 min then filtered and washed over the filter with cold n-heptane.

Yield: 1st crop: 50% wt yield from n-heptane with 98.20% PAR purity by HPLC.
2nd crop: 16.7% wt yield from n-heptane with 99.00% PAR purity by HPLC.
Combined yield: 66.7% by wt.
Purity: Up to 99.0% PAR purity by HPLC.
Appearance: White solid.
Stable to light and air at ambient temperature.

¹H NMR (300 MHz, CDCl₃): δ=0.88 (3H, t, J=6.5 Hz), 0.92 (3H, s), 1.10 (3H, s), 1.18 (3H, s), 1.23-1.37 (4H, m), 1.37-1.47 (2H, m), 1.41 (3H, s), 1.55-1.74 (3H, m), 1.70 (3H, br s), 1.88-2.13 (7H, m), 2.39-2.56 (4H, m), 3.23 (1H, d, J=14.9 Hz), 3.43 (1H, d, J=9.6 Hz), 3.88 (1H, d, J=14.9 Hz), 6.18 (1H, br s), 6.61 (1H, s), 6.68 (H, s).

¹³C NMR (75 MHz; CD₃OD): δ=13.95, 19.24, 19.64, 20.04, 22.44, 23.31, 24.83, 25.08, 26.82, 27.34, 30.50, 31.03, 31.36, 34.10, 35.30, 42.39, 42.86, 45.51, 47.78, 48.19, 58.14, 77.67, 114.31, 116.22, 116.42, 123.26, 134.78, 142.92, 147.53, 154.90, 213.86.

LCMS ESI m/z [MH⁺] 529. DSC 96.50 to 104.55° C.

### EXAMPLE 2

### Δ9-THC 2-thiophene sulfonate

To a solution of crude Δ9-THC (20 g, 63.6 mmol) and Et₃N (8.36 g, 82.68 mmol) in toluene (6 vols) was added solid 2-thiophene sulfonyl chloride (14.52 g, 79.5 mmol) under a N₂ blanket. TLC monitoring of reaction progress showed incomplete reaction after stirring for 3 days at 23° C. More Et₃N (0.33 molar equivalent) and 2-thiophene sulfonyl chloride (0.2 molar equivalent) were added and stirring was continued for 22 h at 40° C. The crude Δ9-THC sulfonate derivative was isolated by standard aqueous work up as described for Example 1 [More toluene (20 vols) was used in this case due to the formation of an emulsion, and the aqueous phase was further re-extracted with MTBE (20 vols) to recover more material]. The combined organic phases were dried over MgSO₄ and filtered. The solvent was evaporated to dryness under reduced pressure. The crude residue was triturated with MeOH (5 vols) to induce crystallisation. The brownish pale slurry was further stirred at 0° C. for 30 min then filtered and washed over the filter with cold MeOH (1.5 vols).

Yield: 1st crop: 71.2% wt yield from MeOH with 99.40% PAR purity by HPLC.
2nd crop: 9.6% wt yield from MeOH with 98.2% PAR purity by HPLC.
Combined yield: 80.8% by wt.
Purity: Up to 99.40% PAR purity by HPLC.
Appearance: Pale solid.
Stability: Stable to light and air at ambient temperature.

¹H NMR (300 MHz, CDCl₃): δ=0.88 (3H, t, J=6.8 Hz), 0.94 (3H, s), 1.19-1.36 (5H, m), 1.37 (3H, m), 1.40-1.70 (3H, m), 1.67 (3H, br s), 1.80-1.90 (1H, m), 2.10-2.20 (2H, m), 2.43 (2H, t, J=7.6 Hz), 2.94 (1H, br d, J=10.3 Hz), 6.17 (1H, m), 6.39 (1H, br s), 6.56 (1H, br s), 7.09 (1H, m), 7.60 (1H, dd, J=3.8 Hz, 1.3 Hz), 7.70 (1H, dd, J=5.0 Hz, 1.3 Hz).

¹³C NMR (75 MHz; CD₃OD): δ=13.97, 19.16, 22.45, 23.32, 24.81, 27.36, 30.44, 31.04, 31.29, 33.82, 35.20, 45.29, 77.52, 114.49, 116.06, 116.51, 123.14, 127.44, 134.28, 134.51, 135.05, 135.69, 142.67, 148.19, 154.63.

LCMS ESI m/z [MH⁺] 461. DSC 92.6 to 96.9° C.

### EXAMPLE 3

### Δ9-THC n-propyl sulfonate

To a solution of crude Δ9-THC (5.33 g, 16.94 mmol) and Et₃N (1.88 g, 18.64 mmol) in toluene (5 vols) was added neat n-propyl sulfonyl chloride (2.65 g, 18.64 mmol) under inert conditions (N₂ blanket). The temperature rose from 22° C. to 35° C. towards the end of addition. The resulting slurry was stirred for 19 h at 18° C. to 22° C. More Et₃N (0.5 molar equivalent) and n-propyl sulfonyl chloride (0.5 molar equivalent) were added to the reaction mixture at ambient temperature and the reaction progress was monitored by TLC. After stirring for 5 h the reaction was quenched by the addition of water (8 vols) followed by stirring for 30 min. The toluene phase was separated and further washed with water (2×8 vols) and saturated aqueous NaCl (1×8 vols). The organic phase was dried over MgSO₄ and filtered. The solvent was evaporated to dryness under reduced pressure. The crude residue was triturated with MeOH (5 vols) to induce crystallisation. The brownish crude slurry was further stirred at −10° C. to −15° C. then filtered and washed over the filter with cold MeOH (2 vols).

Yield: 1^{st} crop: 46.8% by wt with 98.46% PAR purity by HPLC.
2^{nd} crop: 38.5% by wt with 97.83% PAR purity by HPLC.
Combined yield: 85.3% by wt.
Purity: Up to 98.46% PAR purity by HPLC.
Appearance: Pale solid.
Stability: Stable to light and air at ambient temperature.

¹H NMR (300 MHz, CDCl₃): δ=0.88 (3H, t, J=6.7 Hz), 1.09 (3H, s), 1.13 (3H, t, J=7.45 Hz), 1.29-1.47 (5H, m), 1.41 (3H, s), 1.52-1.68 (3H, m), 1.68 (3H, s), 1.88-1.94 (1H, m), 2.01-2.09 (2H, m), 2.14-2.16 (2H, m), 2.50 (2H, t, J=7.5 Hz), 3.23-3.39 (2H, m), 3.40 (1H, br d, J=10.7 Hz), 6.12 (1H, br d, J=1.15 Hz), 6.60 (1H, s), 6.63 (1H, s).

¹³C NMR (75 MHz; CD₃OD): δ=12.91, 13.94, 17.25, 19.20, 22.24, 23.32, 24.80, 27.32, 29.65, 31.03, 31.26, 34.03, 35.29, 45.53, 52.86, 77.65, 114.30, 116.18, 116.35, 123.39, 134.67, 142.87, 147.40, 154.91.

LCMS ESI m/z [MH⁺] 421. DSC 54.77 to 59.38° C.

### EXAMPLE 4

### Δ9-THC 3-chloropropyl sulfonate

To a solution of crude Δ9-THC, triethylamine and 4-dimethylaminopyridine in toluene at 22° C., was added a solution of 3-chloropropyl sulfonyl chloride in toluene over a period of 10 min. The temperature of the resultant suspension was kept below 30° C. during the addition. The reaction progress was monitored by TLC. After stirring for 16 h at 25° C. to 30° C. the mixture was filtered through silica gel and the silica gel washed with toluene. The combined filtrates were concentrated under reduced pressure, methanol was added and distillation continued until the solvent was exchanged to methanol. The solution was cooled to −18° C. and the solid produced was filtered and washed with methanol to give the title compound as a pale yellow solid.
Yield: 83.4% yield by wt.
Purity: 99.1% PAR purity by HPLC.
Appearance: Pale solid.
Stability: Stable to light and air at ambient temperature.

¹H NMR (300 MHz, CDCl₃): δ=0.88 (3H, t, J=6.7 Hz), 1.10 (3H, s), 1.25-1.36 (4H, m), 1.36-1.48 (4H, m), 1.42 (3H, s), 1.52-1.65 (2H, m), 1.67-1.75 (4H, m), 1.69 (3H, s), 1.86-1.96 (1H, m), 2.12-2.22 (2H, m), 2.44-2.57 (4H, m), 3.35-3.85 (3H, m), 3.67-3.82 (2H, m), 6.12 (1H, s), 6.63 (1H, s), 6.67 (1H, s).

¹³C NMR (75 MHz; CD₃OD): δ=13.95, 19.21, 22.44, 23.35, 24.78, 26.71, 27.32, 30.48, 31.02, 31.36, 34.00, 35.28, 42.43, 45.53, 48.43, 77.73, 114.21, 116.20, 116.58, 123.23, 134.97, 143.02, 147.29, 154.98.

LCMS ESI m/z [MH⁺] 455/457. DSC 54.43 to 58.37° C.

### EXAMPLE 5

### Regeneration of Δ9-THC from Δ9-THC 2-thiophene sulfonate

To Δ9-THC 2-thiophene sulfonate (10 g, 21.7 mmol) is added water (5 ml) followed by a solution of potassium t-butoxide in t-butanol (1M, 44 ml) over a period of 15 minutes. The reaction is heated at about 60° C. to 70° C. for 8 hours, cooled and water (25 ml) added. The solution is extracted with heptane (3×50 ml), the organic extracts combined, washed with water (100 ml), brine (100 ml), dried over MgSO₄ and evaporated to give Δ9-THC.

The invention thus provides Δ9-THC derivatives and their uses, e.g. for storage and purification of Δ9-THC.

Accordingly, one aspect of the invention provides a process for the purification of Δ9-THC, which comprises:
(i) preparing a sulfonate derivative of Δ9-THC of formula (I) as herein described;
(ii) purifying the compound of formula (I); and
(iii) hydrolysing the compound of formula (I) to regenerate Δ9-THC.

In a preferred embodiment the purification step comprises recrystallisation of the compound of formula (I). In another preferred embodiment the purification step comprises column chromatography.

Purification of the compounds of formula (I) may comprise one or more of the above methods.

In another aspect the invention provides a process for the preparation of pharmaceutical composition comprising Δ9-THC, which comprises purification of Δ9-THC as herein described followed by formulating the Δ9-THC with a pharmaceutically acceptable diluent or carrier. In a preferred embodiment the pharmaceutical composition comprises sesame seed oil in soft gelatine capsules.

1. A compound of formula (I) 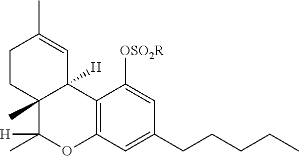
wherein R is selected from
(i) a 5 to 7 membered heterocyclic ring containing 1 to 3 heteroatoms independently selected from nitrogen, oxygen and sulfur, which is optionally substituted by one or more groups independently selected from halo, CN, NO₂, (C₁-C₆)alkyl, (C₁-C₆)alkoxy and phenyl;
(ii) (C₁-C₆)alkyl, which is optionally substituted by one or more substituents each independently selected from halo, oxo, CN, NO₂, (C₁-C₆)alkyl, (C₁-C₆)alkoxy and phenyl;
(iii) (C₁-C₆)alkyl substituted by (C₃-C₁₃)cycloalkyl, which is optionally substituted by one or more substituents each independently selected from halo, oxo, CN, NO₂, (C₁-C₆)alkyl, (C₁-C₆)alkoxy and phenyl; and
(iv) (C₃-C₁₃)cycloalkyl, which is optionally substituted by one or more substituents each independently selected from halo, oxo, CN, NO₂, (C₁-C₆)alkyl, (C₁-C₆)alkoxy and phenyl.

2. The compound of claim 1 wherein R is a 5 to 6 membered heteroaromatic ring containing 1 to 2 heteroatoms independently selected from nitrogen, oxygen and sulfur, which is optionally substituted by one or more groups independently selected from halo, (C₁-C₆)alkyl and phenyl.

3. The compound of claim 2 wherein R is a 5 membered heteroaromatic ring containing 1 heteroatom selected from nitrogen, oxygen and sulfur, which is optionally substituted by one or more groups independently selected from halo, (C₁-C₆)alkyl and phenyl.

4. The compound of claim 3 wherein R is selected from thienyl, furyl and pyrrolyl, which is optionally substituted by one or more groups independently selected from halo, (C₁-C₆)alkyl and phenyl.

5. The compound of claim 4 wherein R is selected from thienyl, furyl and pyrrolyl.

6. The compound of claim 5 wherein R is 2-thienyl.

7. The compound of claim 1 wherein R is (C₁-C₆)alkyl, which is optionally substituted by one or more substituents each independently selected from halo, oxo, (C₁-C₆)alkyl and phenyl.

8. The compound of claim 7 wherein R is (C₁-C₆)alkyl, which is optionally substituted by one or more chloro substituents.

9. The compound of claim 8 wherein R is n-propyl, which is optionally substituted by chloro.

10. The compound of claim 9 wherein R is n-propyl or 3-chloropropyl.

11. The compound of claim 1 wherein R is (C₃-C₁₃)cycloalkyl or (C₁-C₆)alkyl substituted by (C₃-C₁₃)cycloalkyl, each cycloalkyl being optionally substituted by one or more substituents each independently selected from halo, oxo and (C₁-C₆)alkyl.

12. The compound of claim 11 wherein R is (C₃-C₁₀)cycloalkyl or methyl substituted by (C₃-C₁₀)cycloalkyl, each cycloalkyl being optionally substituted by 1 to 3 substituents each independently selected from chloro, oxo and methyl.

13. The compound of claim 12 wherein R is methyl substituted by cyclopropyl, cyclobutyl, cyclopentyl, cyclohexyl, norbornyl, decalinyl or adamantyl, which is optionally substituted by 1 to 3 substituents each independently selected from chloro, oxo and methyl.

14. The compound of claim 13 wherein R is camphor.

15. The compound of claim 1, which is selected from:
Δ9-THC (1S)-(+)-Camphorsulfonate;
Δ9-THC 2-thiophene sulfonate;
Δ9-THC n-propyl sulfonate; and
Δ9-THC 3-chloropropyl sulfonate.

16. A process for the purification of Δ9-THC, which comprises:
preparing the compound of formula (I) of claim 1;
(ii) purifying the compound of formula (I); and
(iii) hydrolysing the compound of formula (I) to regenerate Δ9-THC.

17. The process of claim 16 wherein the purification step comprises recrystallisation of the compound of formula (I).

18. The process of claim 16 wherein the purification step comprises column chromatography.

19. A process for the preparation of a pharmaceutical composition comprising Δ9-THC, which comprises purification of Δ9-THC according to the process of claim 16 followed by formulating the Δ9-THC with a pharmaceutically acceptable diluent or carrier.

20. The process of claim 19 wherein the pharmaceutical composition comprises sesame seed oil in soft gelatine capsules.

21. A process for the preparation of a compound of formula (I) according to claim 1, which comprises reacting Δ9-THC with a sulfonyl halide of formula RSO₂Cl in the presence of a base.
